(19) United States
(12) Patent Application Publication
(10) Pub. No.: US 2004/0033567 A1
(43) Pub. Date: Feb. 19, 2004

(54) Hydrolysed N-source

(75) Inventors: Mogens Wumpelmann, Herlev (DK); Niels Banke, Soborg (DK); Soren Michaelsen, Malov (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 10/603,723

(22) Filed: Jun. 24, 2003

Related U.S. Application Data
(60) Provisional application No. 60/430,419, filed on Dec. 2, 2002.
(60) Provisional application No. 60/393,275, filed on Jul. 1, 2002.

(30) Foreign Application Priority Data
Jul. 1, 2002 (DK) PA 2002 01021
Nov. 28, 2002 (DK) PA 2002 01838

(51) Int. Cl. C12P 21/04

(52) U.S. Cl. 435/71.2

(57) ABSTRACT

A method for the production of an enzyme of interest, on an industrial scale, comprising

a) fermentation of a microbial strain producing an enzyme of interest in a fermentation medium comprising one or more partially prehydrolysed complex N-source(s), wherein said partially prehydrolysed N-source(s) are sterilised separately from any other source containing carbohydrates, the prehydrolysis being achieved by addition of an acid and/or a hydrolytic enzyme; and

b) recovery of the enzyme of interest from the fermentation broth.

### TECHNICAL FIELD

The present invention relates to a method of fermenting an enzyme of interest in a more economical way by adding one or more partially prehydrolysed complex N-sources to the fermentation medium.

### BACKGROUND ART

The media used for fermentative production of valuable compounds on an industrial scale contain normally traditional N-sources such as soy, or corn steep liquor, or yeast extracts. The drawbacks by using these traditional N-sources are high viscosity, raw material variation, problematic recovery, formation of coloured substances during heat sterilisation or that the N-source is too costly or used too fast.

Alternatively to the traditional N-sources, minimal media may be used, e.g. as suggested in WO 98/37179, but the drawbacks here are slow outgrowth and low yields.

WO 01/05997 describes production of Tetanus Toxin by using a media comprising hydrolyzed soy; the inventors state on page 67 that autoclaving glucose with the rest of the medium is beneficial for seed growth and toxin production.

### SUMMARY OF THE INVENTION

The inventors have found that in order to satisfy the amino acid/peptide requirements for fast outgrowth of the microbial strain of interest and/or for achieving high productivities of the product of interest, a partially prehydrolysed complex N-source should be added to the fermentation broth, so we claim:

A method for the production of an enzyme of interest, on an industrial scale, comprising

a) fermentation of a microbial strain producing an enzyme of interest in a fermentation medium comprising one or more partially prehydrolysed complex N-source(s), wherein said partially prehydrolysed N-source(s) are sterilised separately from any other source containing carbohydrates, the prehydrolysis being achieved by addition of an acid and/or a hydrolytic enzyme; and

b) recovery of the enzyme of interest from the fermentation broth.

### DETAILED DISCLOSURE OF THE INVENTION

Microorganisms

The microorganism (the microbial strain) according to the invention may be obtained from microorganisms of any genus.

In a preferred embodiment, the enzyme of interest may be obtained from a bacterial or a fungal source.

For example, the enzyme of interest may be obtained from a gram positive bacterium such as a Bacillus strain, e.g., Bacillus alkalophilus, Bacillus amyloliquefaciens, Bacillus brevis, Bacillus circulans, Bacillus coagulans, Bacillus lautus, Bacillus lentus, Bacillus licheniformis, Bacillus megaterium, Bacillus stearothermophilus, Bacillus subtilis, or Bacillus thuringiensis; or a Streptomyces strain, e.g., Streptomyces lividans or Streptomyces murinus; or from a gram negative bacterium, e.g., E. coli or Pseudomonas sp.

The enzyme of interest may be obtained from a fungal source, e.g. from a yeast strain such as a Candida, Kluyveromyces, Pichia, Saccharomyces, Schizosaccharomyces, or Yarrowia strain, e.g., Saccharomyces carlsbergensis, Saccharomyces cerevisiae, Saccharomyces diastaticus, Saccharomyces douglasii, Saccharomyces kluyveri, Saccharomyces norbensis or Saccharomyces oviformis strain.

The enzyme of interest may be obtained from a filamentous fungal strain such as an Acremonium, Aspergillus, Aureobasidium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, or Trichoderma strain, in particular the enzyme of interest may be obtained from an Aspergillus aculeatus, Aspergillus awamori, Aspergillus foetidus, Aspergillus japonicus, Aspergillus nidulans, Aspergillus niger, Aspergillus oryzae, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Trichoderma harzianum, Trichoderma koningii, Trichoderma longibrachiatum, Trichoderma reesei, or Trichoderma viride strain.

Strains of these species are readily accessible to the public in a number of culture collections, such as the American Type Culture Collection (ATCC), Deutsche Sammlung von Mikroorganismen und Zellkulturen GmbH (DSM), Centraalbureau Voor Schimmelcultures (CBS), and Agricultural Research Service Patent Culture Collection, Northern Regional Research Center (NRRL).

For purposes of the present invention, the term “obtained from” as used herein in connection with a given source shall mean that the enzyme of interest is produced by the source or by a cell in which a gene from the source has been inserted.

Enzyme of Interest

The enzyme of interest may be a peptide or an enzyme.

A preferred peptide according to this invention contains from 5 to 100 amino acids; preferably from 10 to 80 amino acids; more preferably from 15 to 60 amino acids; even more preferably from 15 to 40 amino acids.

In a preferred embodiment, the method is applied to enzymes, in particular to hydrolases (class EC 3 according to Enzyme Nomenclature; Recommendations of the Nomenclature Committee of the International Union of Biochemistry).

In a particular preferred embodiment the following hydrolases are preferred:

Proteases:

Suitable proteases include those of animal, vegetable or microbial origin. Microbial origin is preferred. Chemically modified or protein engineered mutants are included. The protease may be an acid protease, a serine protease or a metallo protease, preferably an alkaline microbial protease or a trypsin-like protease.

Proteases and peptidases are defined as being self-destructive and non-destructive if >= or <10%, respectively, of the enzymatic activity in the cell free culture broth at the preferred harvest time has disappeared upon incubation for 24 h of the cell free culture broth at the pH and temperature values selected in the fermentation process, these values being representative for the pH and temperature range imposed during the fermentation process from 24 h before harvest and until harvest of the broth.

Cell free culture broth is produced from the culture broth by filtration, centrifugation or similar processes separating insoluables (incl. cells) from the soluables in the broth.

Examples of alkaline proteases are subtilisins, especially those derived from Bacillus, e.g., subtilisin Novo, subtilisin Carlsberg, subtilisin 309, subtilisin 147 and subtilisin 168 (described in WO 89/06279). Examples of trypsin-like proteases are trypsin (e.g. of porcine or bovine origin) and the Fusarium protease described in WO 89/06270 and WO 94/25583.

Examples of useful proteases are the variants described in WO 92/19729, WO 98/20115, WO 98/20116, and WO 98/34946, especially the variants with substitutions in one or more of the following positions: 27, 36, 57, 76, 87, 97, 101, 104, 120, 123, 167, 170, 194, 206, 218, 222, 224, 235 and 274.

Preferred commercially available protease enzymes include ALCALASE™, SAVINASE™, PRIMASE™, DURALASE™, ESPERASE™, RELASE™ and KANNASE™ (Novozymes A/S), MAXATASE™, MAXACAL™, MAXAPEM™, PROPERASE™, PURAFECT™, PURAFECT OXP™, FN2™, and FN3™ (Genencor International Inc.).

Peptidases:

An example of a suitable peptidase is FLAVOURZYME™ (Novozymes A/S).

Lipases:

Suitable lipases include those of bacterial or fungal origin. Chemically modified or protein engineered mutants are included. Examples of useful lipases include lipases from Humicola (synonym Thermomyces), e.g. from H. lanuginosa (T. lanuginosus) as described in EP 258 068 and EP 305 216 or from H. insolens as described in WO 96/13580, a Pseudomonas lipase, e.g. from P. alcaligenes or P. pseudoalcaligenes (EP 218 272), P. cepacia (EP 331 376), P. stutzeri (GB 1,372,034), P. fluorescens, Pseudomonas sp. strain SD 705 (WO 95/06720 and WO 96/27002), P. wisconsinensis (WO 96/12012), a Bacillus lipase, e.g. from B. subtilis (Dartois et al. (1993), Biochemica et Biophysica Acta, 1131, 253-360), B. stearothermophilus (JP 64/744992) or B. pumilus (WO 91/16422).

Other examples are lipase variants such as those described in WO 92/05249, WO 94/01541, EP 407 225, EP 260 105, WO 95/35381, WO 96/00292, WO 95/30744, WO 94/25578, WO 95/14783, WO 95/22615, WO 97/04079 and WO 97/07202.

Preferred commercially available lipase enzymes include LIPOLASE™, LIPOLASE ULTRA™ and LIPEX™ (Novozymes A/S).

Amylases:

Suitable amylases (α and/or β) include those of bacterial or fungal origin. Chemically modified or protein engineered mutants are included. Amylases include, for example, α-amylases obtained from Bacillus, e.g. a special strain of B. licheniformis, described in more detail in GB 1,296,839.

Examples of useful amylases are the variants described in WO 94/02597, WO 94/18314, WO 96/23873, and WO 97/43424, especially the variants with substitutions in one or more of the following positions: 15, 23, 105, 106, 124, 128, 133, 154, 156, 181, 188, 190, 197, 202, 208, 209, 243, 264, 304, 305, 391, 408, and 444.

Commercially available amylases are DURAMYL™, TERMAMYL™, FUNGAMYL™, NATALASE™, TERMAMYL LC™, TERMAMYL SC™, LIQUIZYME-X™ and BAN™ (Novozymes A/S), RAPIDASE™ and PURASTAR™ (from Genencor International Inc.).

Cellulases:

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g. the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259.

Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307 and PCT/DK98/00299.

Commercially available cellulases include CELLUZYME™, and CAREZYME™ (Novozymes A/S), CLAZINASE™, and PURADAX HA™ (Genencor International Inc.), and KAC-500(B)™ (Kao Corporation).

Oxidoreductases

Oxidoreductases that may be treated according to the invention include peroxidases, and oxidases such as laccases, and catalases.

Other preferred hydrolases are carbohydrolases including MANNAWAY™ (Novozymes A/S) and pectate lyase (e.g. BIOPREPARATION 3000™ (Novozymes A/S)). Other preferred enzymes are transferases, lyases, isomerases, and ligases.

Complex N-Sources

According to the present invention suitable complex N-sources are proteins of plant or animal origin, in particular proteins of plant or animal origin containing less than 10% of carbohydrate; in particular containing less than 5% of carbohydrate; especially containing less than 3% of carbohydrate.

It is an advantage that the percentage of carbohydrates is low in order to avoid Maillard reactions. Often colour formation (Maillard reactions) during heat sterilization of media from primary amino groups and reducing carbohydrates is highly disadvantageous from perspective of recovery and/or growth inhibition. It is thus important that “partners” in Maillard reactions are separated to a suitable extent during heat sterilization. This implies that separate sterilization of simple carbohydrates (glucose, sucrose, etc.) and complex N-sources should be carried out, and that the complex N-sources should be selected among the sources available that contain a low amount of reducing carbohydrates (e.g. potato protein, pea protein, blood protein, fish protein, animal protein).

However, for someone skilled in the art it is well understood, that the presence of only minor amounts of carbohydrate during the sterilization of the complex N-source will not have a significant effect on either the enzyme recovery process or on the growth. Therefore, separate sterilization of carbohydrate and complex N-sources should imply, that less than 10% of all carbohydrate added during the fermentation is sterilized together with the complex N-source.

It is well understood by someone skilled in the art that the effect of heat sterilization on the amount of reducing carbohydrate in the medium potentially available for Maillard reactions to occur is scale dependent. Thus, the suitability of a certain complex N-source selection in conjuction with the selection of conditions for complex N-source prehydrolysis should be evaluated in production scale.

The amount of prehydrolysed complex N-sources added to the fermentation medium is of at least 5% (w/w) of the total amount of N-Kjeldahl added to the fermentation medium, in particular of 10-75% (w/w) of the total amount of N-Kjeldahl added to the fermentation medium.

Prehydrolysis

Enzymatic prehydrolysis of the complex N-source is preferred, but the invention may also be carried out using other techniques such as acid hydrolysis. Examples of preferred embodiments of prehydrolysis procedures are given.

The desired degree of prehydrolysis is preferably achieved by properly adjusting the hydrolysis temperature, the amount of protease and/or peptidase added, the time allowed for the prehydrolysis to occur and by the selection of hydrolytic enzymes used in the prehydrolysis in conjunction with the selection of proper pH intervals for the prehydrolysis to occur with the hydrolytic enzymes chosen.

The desired degree of prehydrolysis would depend on several factors:

From the perspective of achieving high product titers and thus high volumetric product productivities the use of highly concentrated feed media is potentially advantageous. Thus, adding separately sterilised complex N-sources to the feed medium should be avoided if sufficient amounts of readily utilisable complex N-sources—gradually throughout the fermentation—can be made available from not readily available complex N-sources in the make-up medium present in the fermentor prior to inoculation in order for the biomass formation and/or the product formation to become stimulated. Achieving such continued availability of readily utilisable complex N-sources is the objective of carrying out the prehydrolysis, which then should be adjusted in terms of degree of prehydrolysis achieved in conjunction with the amount of proteases and/or peptidases produced by the strain itself during cultivation.

From the perspective of achieving high specific product productivities—that is, high rates of product formation from individual, active cells an identical argumentation can be applied.

From the perspective of achieving high specific product productivities when the product is an enzyme with the catalytical capability of inactivating itself in uni- or bimolecular reactions the addition of media components protecting against such product self inactivation can be highly advantageous. Complex N-sources can be such protecting media components the effect of which can depend upon when such media components are added to the fermentation broth. Thus, it can be found, that adding such media components to the feed medium is highly advantageous—especially when such media components are prehydrolysed to an extent allowing for such media components being pumpable in large scale equipment while still maintaining highly protective effects.

The term “pumpable” is used to characterise a suspension of solid particles that rarely forms clumps in pumps, valves and piping systems used—the presence of such clumps altering feed rates by more than 5%.

If the enzyme of interest is an amylase, a cellulase, a lipase, an oxidoreductase, a carbohydrolase or a non-destructive protease or peptidase the prehydrolysis is preferably giving rise to breakage of between 10 and 70% of the peptide bonds, more preferably between 15 and 40% of the peptide bonds.

If the enzyme of interest is a self-destructive protease or a peptidase the prehydrolysis is preferably giving rise to breakage of between 1 and 20% of the peptide bonds, more preferably between 2 and 10% of the peptide bonds.

If the enzyme of interest is a self-destructive protease or a peptidase it might be especially advantageous to use as the complex N-source a mixture of highly hydrolysed protein and only slightly hydrolysed protein the preferred degree of prehydrolysis thus stated above for producing such enzymes of interest, for the total amount of complex N-source added, being calculated as:

[DPH(highly hydr.)×W(highly hydr.)+DPH(slightly hydr.)×W(slightly hydr.)]/[W(highly hydr.)+W(slightly hydr.)];

wherein

DPH(highly hydr.) is the degree of prehydrolysis of the highly hydrolysed protein;

DPH(slightly hydr.) is the degree of prehydrolysis of the slightly hydrolysed protein;

W(highly hydr.) is the weight of highly hydrolysed protein used in the medium; and

W(slightly hydr.) is the weight of slightly hydrolysed protein used in the medium.

Fermentations

The present invention may be useful for any fermentation in industrial scale, e.g. for any fermentation having culture media of at least 50 litres, preferably at least 100 litres, more preferably at least 500 litres, even more preferably at least 1000 litres, in particular at least 5000 litres.

The microbial strain may be fermented by any method known in the art. The fermentation medium may be a complex medium comprising complex nitrogen and carbon sources. The fermentation may be performed as a batch, a repeated batch, a fed-batch, a repeated fed-batch or a continuous fermentation process.

In a fed-batch process, either none or part of the compounds comprising one or more of the structural and/or catalytic elements is added to the medium before the start of the fermentation and either all or the remaining part, respectively, of the compounds comprising one or more of the structural and/or catalytic elements is fed during the fermentation process. The compounds which are selected for feeding can be fed together or separate from each other to the fermentation process.

In a repeated fed-batch or a continuous fermentation process, the complete start medium is additionally fed during fermentation. The start medium can be fed together with or separate from the structural element feed(s). In a repeated fed-batch process, part of the fermentation broth comprising the biomass is removed at regular time intervals, whereas in a continuous process, the removal of part of the fermentation broth occurs continuously. The fermentation process is thereby replenished with a portion of fresh medium corresponding to the amount of withdrawn fermentation broth.

In a preferred embodiment of the invention, a fed-batch, a repeated fed-batch process or a continuous fermentation process is preferred.

Recovery of the Valuable Compound

A further aspect of the invention concerns the downstream processing of the fermentation broth. After the fermentation process is ended, the enzyme of interest may be recovered from the fermentation broth, using standard technology developed for the enzyme of interest.

The invention is further illustrated in the following examples which are not intended to be in any way limiting to the scope of the invention as claimed.

### EXAMPLE 1

### Hydrolysis of Potato Protein: OPA=51%

To 3.2 kg potato protein was added tap water to 12.5 liter; this mixture was agitated in order for the potato protein to become fully suspended.

While still agitating heating was applied (set point 54° C.).

When the temperature reached 45° C., pH was adjusted to 6.0 with 4 N NaOH.

When the temperature reached 50° C., 80 ml ALCALASE™ 2.4 L FG (available from Novozymes A/S) was added while pH was maintained at 6.0 by addition of 4 N NaOH. 54° C. was reached shortly (approx. 5 min) after.

10 min after the ALCALASE addition the set point for pH-control was changed from 6.0 to 8.0.

After further 26 min from the ALCALASE addition pH-control by NaOH addition was deactivated and further 1.6 kg potato protein added.

After 3 min of fully suspending the added potato protein 150 ml of FLAVOURZYME™ 1000 L (available from Novozymes A/S) was added.

After 20 h from the addition of ALCALASE tap water was added to 16 liter, and the hydrolysis terminated by transferring the hydrolysed protein in suspension to portions of 4 liter, immediately stored in a −18° C. freezer.

The degree of hydrolysis (OPA) was determined as described in Example 4 assuming a dry matter content in potato protein of 93% and a protein content in potato protein as % of dry matter of 80%.

### EXAMPLE 2

### Hydrolysis of Potato Protein: OPA=2.9%

To 2.09 kg potato protein was added tap water to 10.5 liter; this mixture was agitated in order for the potato protein to become fully suspended.

While still agitating heating was applied (set point 55° C.).

When the temperature reached 30° C., pH was adjusted to 6.2 with 4 N NaOH.

When the temperature reached 55° C., 58.5 ml ALCALASE™ 2.4 L FG was added while pH was maintained at 6.2 by addition of 4 N NaOH.

5 min after the ALCALASE addition the set point for pH-control was changed from 6.2 to 8.0.

After further 30 min from the ALCALASE addition pH was manually lowered over 5 min to 5.6 by 15% H3PO4 addition and further 1.575 kg potato protein added.

Immediately after, tap water was added to 15 liter and the hydrolysis terminated by transferring the hydrolysed protein in suspension to portions of 2 liter, immediately stored in a −18° C. freezer.

The degree of hydrolysis (OPA) was determined as described in Example 4 assuming a dry matter content in potato protein of 93% and a protein content in potato protein as % of dry matter of 80%.

### EXAMPLE 3

### Hydrolysis of Potato Protein: OPA=19.5%

To 1.2 kg potato protein was added tap water to 13 liter; this mixture was agitated in order for the potato protein to become fully suspended.

While still agitating heating was applied (set point 55° C.).

When the temperature reached 55° C., pH was adjusted to 7.0 with 4 N NaOH and 116.6 g ALCALASE™ 2.4 L FG added while pH was maintained at 7.0 by addition of 4 N NaOH.

4 h after the ALCALASE addition, tap water was added to 16 liter and the hydrolysis terminated by transferring the hydrolysed protein in suspension to portions of 4 liter, immediately stored in a −18° C. freezer.

The degree of hydrolysis (OPA) was determined as described in Example 4 assuming a dry matter content in potato protein of 93% and a protein content in potato protein as % of dry matter of 80%.

### EXAMPLE 4

### Analytical Determination of OPA, the Degree of Protein Hydrolysis

Approx. 1 g of sample (weight of sample=W1) was mixed with 4 ml 0.1 N NaOH.

The mixture was centrifuged until the supernatant was clear. The supernatant was then appropriately diluted with deionised water (to V1 ml).

3 ml OPA reagent (see below) was then added at time zero and the mixture vortexed (mixed intensively). OD (340 nm, 1 cm cuvette) was measured after exactly 2 min.

Duplicates were made for each sample.

The average OD must be between OD measured for blind and standard; otherwise the dilution was changed accordingly.

Blind: Deionised Water

Standard: 50 mg L-serine; add deionised water to 500 ml.

OPA Reagent:

Weigh out 7.62 g disodium tetraborate+200 mg SDS; add deionised water to approx. 175 ml. Add 160 mg ortho-phthaldialdehyde (OPA) to 4 ml 96% EtOH and solubilise. Add solubilised OPA to borax/SDS solution. Further add 176 mg dithiothreitol (99%) and finally adjust volume to 200 ml with deionised water. Discard OPA reagent after 4 hours.

OPA (degree of hydrolysis) was calculated as:

((A×(ODav.,sample−ODav.,blind)/(ODav.,standard−ODav.,blind) ×(V1(ml)×100)/(W1(mg)×P))−B)×100%/(C×D)

A=0.9516=concentration of the serine standard meqv/L

ODav.,sample=the average OD(340 nm) value measured for the sample

ODav.,standard=the average OD(340 nm) value measured for the serine standard

ODav.,blind=the average OD(340 nm) value measured for the blind

V1 (ml)=dilution volume in mL

W1 (mg)=sample in mg

P=% potato protein in the hydrolysis sample

B=0.4, constant chosen for potato protein

C=1.0, constant chosen for potato protein

D=9.1, constant chosen for potato protein

B, C, D values for other protein types:

| Protein | B | C | D |
|---|---|---|---|
| Soya | 0.342 | 0.97 | 7.8 |
| Gluten | 0.4 | 1.0 | 8.3 |
| Casein | 0.383 | 1.039 | 8.2 |
| Meat | 0.4 | 1.0 | 7.6 |
| Fish | 0.4 | 1.0 | 8.6 |
| Other | 0.39 | 1.0 | 8.5 |

The OPA value is thus reflecting the percentage of peptide bonds hydrolysed within the sample analysed.

### EXAMPLE 5

### Strains

The protease strain used in Example 6 (Af50-34) and further used in Example 7 and 8 was an isolate of NCIB 10309 and genetically modified as described in EP 0 506 780 B1.

The alpha-amylase strain used in Example 6 (SJ 5262) and further used in Example 9 and 10 was derived from strain SJ4671 described in U.S. Pat. No. 6,100,063. In a first step, a spontaneous rifampicin-resistant mutant was isolated which contained a substitution of amino acid number 478 in the RpoB protein from alanine to valine, resulting in strain SJ4671 rif10 disclosed in the copending Danish patent application PA 2001 01972. In a second step the gene encoding an extracellular protease (protein and DNA sequence published in GeneSeqP accession no: AAE00011; WO 01/16285; EP 482 879) was deleted from the chromosome by double homologous recombination by the general procedure described in WO 02/00907.

### EXAMPLE 6

### Propagation Procedures Used

The Af50-34 strain:

| B3-apar: | | |
|---|---|---|
| Peptone | 6 | g |
| Pepticase | 4 | g |
| Yeast extract | 3 | g |
| Meat extract | 1.5 | g |
| Glucose.1H2O | 1 | g |
| Agar | 20 | g |

| | |
|---|---|
| Deionised water | added to 1 l after pH adjustment |
| | to 7.35 with NaOH/HCl. |

Sterilised at 121° C. for 40 min.

After cooling to 40-50° C., 10% v/v of 1M NaHCO3, pH 9, sterilised by filtration and 10% v/v of 10% w/v dried skim milk in deionised water, sterilised at 121° C. for 40 min, was added.

| M9-buffer: | | |
|---|---|---|
| Na2HPO4.2H2O | 8.8 | g |
| KH2PO4 | 3 | g |
| NaCl | 4 | g |
| MgSO4.7H2O | 0.2 | g |
| Deionised water | add to 1 liter | |
| Sterilised at 121° C. for 20 min. | | |
| Seed shake flask medium: | | |
| PRK-1: | | |
| Soya | 50 | g |
| Na2HPO4.2 H2O | 20 | g |

| | |
|---|---|
| Deionised water | added to 1 l after pH adjustment |
| | to 9.0 with NaOH/HCl. |

Sterilised at 121° C. for 20 min; 100 ml in 500 ml
conical flasks with 2 baffles.

The strain (Af50-34) was incubated on B3-agar slants for 24 h at 37° C.

The biomass thus produced was then suspended in M9-buffer. OD (650 nm) of this suspension was measured. A volume, y ml of the cell suspension (OD(650 nm)x y=0.1) was used for inoculating each PRK-1 shake flask, incubated at 37° C. for 22 h at 300 rpm on a HT Infors Unitson rotating shaker.

80 ml of this shake flask culture broth was used for inoculating each fermentor.

The SJ 5262 strain:

| LB-agar: | | |
|---|---|---|
| Peptone from casein | 10 | g |
| Yeast extract | 5 | g |
| NaCl | 10 | g |
| Agar | 12 | g |
| Deionised water | added to 1 liter after pH | |
| | adjustment to 7 (+/−0.2) with | |
| | NaOH/HCl. | |
| Sterilised at 121° C. for 20 min. | | |
| M9-buffer: | | |
| Na2HPO4.2H2O | 8.8 | g |
| KH2PO4 | 3 | g |
| NaCl | 4 | g |
| MgSO4.7H2O | 0.2 | g |
| Deionised water | added to 1 liter | |
| Sterilised at 121° C. for 20 min | | |
| Seed shake flask medium: | | |
| PRK-50: | | |
| Soy flakes | 44 | g |
| Na2HPO4.2H2O | 2 | g |

Tap water added to 1 liter after pH adjustment to 8.0
with NaOH/HCl.
Sterilised at 121° C. for 60 min; 100 ml in 500 ml
conical flasks with 2 baffles.

The strain (SJ 5262) was incubated on LB-agar slants for 24 h at 37° C.

The biomass thus produced was then suspended in M9-buffer. OD(650 nm) of this suspension was measured. A volume, y ml of the cell suspension (OD(650 nm)x y=0.1) was used for inoculating each PRK-50 shake flask, incubated at 37° C. for 20 h at 300 rpm on a HT Infors Unitson rotating shaker.

80 ml of this shake flask culture broth was used for inoculating each fermentor.

### EXAMPLE 7

### F rmentation with the Af50-34 Strain; Potato Protein with OPA=2.9% in the Feed Medium

The fermentation was carried out in 2 liter fermentors equipped with 4 baffles at agitation and aeration rates sufficient to maintain a dissolved oxygen concentration at or above 20% of saturation throughout. The aeration did not at any time exceed 2 l/l/min.

The temperature was maintained at 37° C. Antifoam oil—in amounts sufficient to prevent foaming becoming uncontrollable - was added initially to the make-up and the feed medium.

pH was maintained between 8.0 and 7.7 by addition of 15% H3PO4 and/or 10% NH3 in water.

Feeding medium was initiated at time 0.1 h from inoculation and was maintained at the following rates:

| Time from feed start (h): | 0 | 10 | 200 |
|---|---|---|---|
| Feed rate (g/min): | 0 | 0.2 | 0.2 |

Make-up medium:

| | | |
|---|---|---|
| Potato protein hydrolysate; OPA = 2.9% | 100 | g |
| KH2PO4 | 5 | g |
| Na2HPO4.2H2O | 5 | g |
| MgSO4.7H2O | 2.5 | g |
| MnSO4.1H2O | 0.02 | g |
| FeSO4.7H2O | 0.08 | g |
| CuSO4.5H2O | 0.008 | g |
| ZnCl2 | 0.008 | g |
| Citric acid | 0.39 | g |
| ThiamineCl2 | 0.05 | g |
| Riboflavin | 0.004 | g |
| Nicotinic acid | 0.03 | g |
| Ca D-pantothenate | 0.04 | g |
| Pyridoxal.HCl | 0.008 | g |
| D-biotin | 0.0015 | g |
| Folic acid | 0.004 | g |

| | |
|---|---|
| Tap water added to | 1.0 liter after pH- |
| | adjustment to 8 with |
| | H3PO4/NH3. |

Sterilised in situ (720 ml/fermentor) at 121° C. for 1 h.

Feed Medium:

| | | |
|---|---|---|
| Potato protein hydrolysate; OPA = 2.9% | 135 | g |
| Sucrose | 300 | g |
| Tap water added to 1.0 liter. | | |
| Sterilised at 121° C. for 1 h | | |

The fermentation was sampled at 49 h and at 71 h from inoculation and samples analysed for protease activity according to Example 11.

### EXAMPLE 8

### Fermentation with the Af50-34 Strain; Potato Protein with OPA=51% in the Feed Medium

This fermentation was carried out exactly as the fermentation described in Example 7 except that potato protein hydrolysate, OPA=51%, was used in the feed medium in amounts equivalent to the amount of protein hydrolysate used in Example 7 when based on dry matter derived from potato protein present in the hydrolysate (110 g hydrolysate/l).

### EXAMPLE 9

### Fermentation with the SJ 5262 Strain; Potato Protein with OPA=19.5% in the Make-Up Medium

The fermentation was carried out in 2 liter fermentors equipped with 4 baffles at agitation and aeration rates sufficient to maintain a dissolved oxygen concentration at or above 20% of saturation throughout. The aeration did not at any time exceed 2 l/l/min.

The temperature was maintained at 37° C. Antifoam oil—in amounts sufficient to prevent foaming becoming uncontrollable—was added initially to the make-up and the feed medium.

pH was maintained between 7.5 and 7.0 by addition of 15% H3PO4 and/or 10% NH3 in water.

Feeding medium was initiated at time 0.1 h from inoculation and was maintained at the following rates:

| Time from feed start (h): | 0 | 5 | 200 |
|---|---|---|---|
| Feed rate (g/min): | 0 | 0.15 | 0.15 |

Make-up medium:

| | | |
|---|---|---|
| Potato protein hydrolysate; OPA = 19.5% | 187.5 | g |
| K2SO4 | 5 | g |
| K2HPO4 | 5 | g |
| Na2HPO4.2H2O | 5 | g |
| MgSO4.7H2O | 2.5 | g |
| (NH4)2SO4 | 2.5 | g |
| MnSO4.1H2O | 0.02 | g |
| FeSO4.7H2O | 0.08 | g |
| CuSO4.5H2O | 0.008 | g |
| ZnCl2 | 0.008 | g |
| Citric acid | 0.39 | g |
| Tap water added to | 1.0 | l |

Sterilised in situ (720 ml/fermentor) at 121° C. for 1 h.

Feed Medium:

| | | |
|---|---|---|
| Glucose.1H2O | 400 | g |

Tap water added to 1.0 liter.
Sterilised at 121° C. for 1 h.

The fermentation was sampled at 95 h and at 116 h from inoculation and samples analysed for alfa-amylase activity according to Example 11.

### EXAMPLE 10

### Fermentatiom with the SJ 5262 Strain; Unhydrolysed Potato Protein in the Make-Up Medium

This fermentation was carried out exactly as the fermentation described in Example 9 except that unhydrolysed potato protein was used in the make-up medium in amounts equivalent to the amount of protein hydrolysate used in Example 9 when based on dry matter derived from potato protein present in the hydrolysate/unhydrolysed protein (15 g/l potato protein).

### EXAMPLE 11

### Analytical Determination of Enzyme Activity in Fermentation Broths

The protease enzyme titers (Example 7 and 8) were measured by methods known within the art based on measuring the enzyme activities present in the culture broth samples, e.g., the method for protease activity analysis described in WO 89/06279 (p. 29-31) may be used.

The alpha-amylase enzyme titers (Example 9 and 10) were measured by methods known within the art based on measuring the enzyme activities present in the culture broth samples, e.g., the method for alpha-amylase activity analysis described in WO 95/26397 (p. 9-10) may be used.

### EXAMPLE 12

Comparison of Enzyme Titers Reached in Example 7, 8, 9, and 10

Af50-34/Protease:

Potato protein hydrolysate; OPA=2.9 in feed (Example 7):

relative titer at 49/71 h: 139/130

Potato protein hydrolysate; OPA=51 in feed (Example 8):

relative titer at 49/71 h: 100/68

(All titers relative to yield at 49 h reached in Example 8)

SJ 5262/Alpha-Amylase:

Potato protein hydrolysate; OPA=19.5 in make-up (Example 9):

relative titer at 95/116 h: 111/130

Unhydrolysed potato protein (Example 10):

relative titer at 95/116 h: 100/117

(All titers relative to yield at 95 h in Example 10)

In conclusion it is thus highly advantageous in the fermentation giving rise to the formation of a protease as the enzyme of interest to use as the complex N-source a (potato) protein hydrolysate with a low degree of prehydrolysis making such hydrolysate pumpable—and it is thus highly advantageous in the fermentation giving rise to the formation of an alpha-amylase as the enzyme of interest to use as the complex N-source a (potato) protein hydrolysate with a degree of prehydrolysis sufficiently high for making the complex N-source available for up take and utilisation by the microorganism in a suitable way.

1. A method for the production of an enzyme of interest, on an industrial scale, comprising
a) fermentation of a microbial strain producing an enzyme of interest in a fermentation medium comprising one or more partially prehydrolysed complex N-sources, wherein said partially prehydrolysed N-sources are sterilised separately from any other source containing carbohydrates, the prehydrolysis being achieved by addition of an acid and/or a hydrolytic enzyme; and
b) recovering the enzyme of interest from the fermentation broth.

2. The method according to claim 1, wherein the enzyme of interest is selected from the group consisting of an amylase, a cellulase, a lipase, an oxidoreductase, a carbohydrolase, and a non-destructive protease or peptidase.

3. The method according to claim 1, wherein the enzyme is a self-destructive protease or peptidase.

4. The method according to claim 1, wherein the microbial strain is a bacterium or a fungus.

5. The method according to claim 4, wherein the bacterium is a Bacillus strain.

6. The method according to claim 1, wherein the complex N-sources are proteins of plant origin containing less than 10% of carbohydrate.

7. The method according to claim 1, wherein the complex N-sources are selected from the group consisting of potato protein and pea protein.

8. The method according to claim 1, wherein the complex N-sources are proteins of animal origin containing less than 10% of carbohydrate.

9. The method according to claim 1, wherein the complex N-sources are selected from the group consisting of blood proteins, fish muscle proteins and animal muscle proteins.

10. The method according to claim 2, wherein the prehydrolysis results in a breakage of between 10 and 70% of the peptide bonds.

11. The method according to claim 3, wherein the prehydrolysis results in a breakage of between 1 and 20% of the peptide bonds.

12. The method according to claim 1, wherein the amount of prehydrolysed complex N-sources is added in an amount of at least 5% (w/w) of the total amount of N-Kjeldahl added to the fermentation medium.

13. The method according to claim 1, wherein the fermentation medium is of at least 50 litres.

14. The method according to claim 1, wherein the fermentation occurs via a repeated batch, a fed batch, a repeated fed batch or a continuous process.
